(19) United States
(12) Patent Application Publication
(10) Pub. No.: US 2024/0219589 A1
(43) Pub. Date: Jul. 4, 2024

(54) DIRECT ATTACH RADIATION DETECTOR STRUCTURES INCLUDING A CARRIER BOARD AND METHODS OF FABRICATION THEREOF

(71) Applicant: REDLEN TECHNOLOGIES, INC., Saanichton (CA)

(72) Inventors: Michael AYUKAWA, Victoria (CA); Bernard HARRIS, Vancouver (CA); Krzysztof INIEWSKI, Port Moody (CA)

(21) Appl. No.: 18/602,887

(22) Filed: Mar. 12, 2024

Related U.S. Application Data
(63) Continuation-in-part of application No. 18/158,695, filed on Jan. 24, 2023.
(60) Provisional application No. 63/489,876, filed on Mar. 13, 2023.
(60) Provisional application No. 63/304,805, filed on Jan. 31, 2022.

(51) Int. Cl. G01T 1/24; A61B 6/42; H01L 27/146; H05B 3/22

(52) U.S. Cl. CPC G01T 1/244; A61B 6/4233; A61B 6/4266; H01L 27/14634; H01L 27/1469; H05B 3/22

(57) ABSTRACT

Direct attach radiation detector structures include an application specific integrated circuit (ASIC), at least one radiation sensor located over a front surface of the ASIC, and a carrier board located over a back surface of the ASIC. In various embodiments, the carrier board may include one or more thermal management features that may reduce temperature non-uniformities in the detector structure. In additional embodiments, the carrier board may include one or more features to improve the manufacturability of the radiation detector unit.

### FIELD

The present disclosure relates generally to radiation detectors, and more specifically to direct attach radiation detector structures including a carrier board.

### BACKGROUND

Room temperature pixelated radiation detectors made of semiconductors, such as cadmium zinc telluride (Cd₁₋ₓZnₓTe where 0<x<1, or “CZT”), are gaining popularity for use in medical and non-medical imaging. These applications use the high energy resolution and sensitivity of the radiation detectors.

### SUMMARY

According to an aspect of the present disclosure, a detector structure includes an application specific integrated circuit (ASIC), at least one radiation sensor located over a front surface of the ASIC, and a carrier board located over a back surface of the ASIC,

In one embodiment, the carrier board includes a plurality of thermal vias extending through the carrier board. In another embodiment, the carrier board includes at least one resistive heating element located on or within the carrier board. In another embodiment, the back surface of the ASIC is mounted to a front surface of the carrier board via a plurality of bonding material portions, and the carrier board includes at least one opening extending through the carrier board.

Further embodiments include detector arrays including a plurality of the above-described detector structures, where the radiation sensors of the plurality of detector structures form a continuous detector surface of the detector array.

Further embodiments include X-ray imaging systems including a radiation source configured to emit an X-ray beam, and a detector array including a plurality of the above-described detector structures that are configured to receive the X-ray beam from the radiation source through an intervening space configured to contain an object therein.

Further embodiments include methods of fabricating a detector structure that include aligning an application specific integrated circuit (ASIC) over a carrier board such that at least two peripheral side surfaces of the ASIC are coincident with peripheral side surfaces of the carrier board and a plurality of bonding material portions are disposed between the ASIC and the carrier board, applying a negative pressure through an opening extending through the carrier board, and bonding the ASIC to the carrier board using the bonding material portions.

### BRIEF DESCRIPTION OF THE DRAWINGS

FIG. 1A is a functional block diagram of an X-ray imaging system in accordance with various embodiments of the present disclosure.

FIG. 1B is a schematically illustration of an application specific integrated circuit (ASIC) configured to count X-ray photons detected in each pixel detector within a set of energy bins according to various embodiments of the present disclosure.

FIG. 2 is a perspective view of a detector array for a computed tomography (CT) X-ray imaging system according to various embodiment of the present disclosure.

FIG. 3A is a vertical cross-sectional view of a radiation detector unit according to an embodiment of the present disclosure.

FIG. 3B is a plan view illustrating the front side of the application specific integrated circuit (ASIC) of the radiation detector unit of FIG. 3A.

FIG. 4A is a vertical cross-section view of an alternative configuration of a radiation detector unit according to an embodiment of the present disclosure

FIG. 4B is a plan view of the ASIC of the radiation detector unit of FIG. 4A.

FIG. 5 is a perspective view of a detector module including a plurality of radiation detector units mounted to frame bar according to an embodiment of the present disclosure.

FIG. 6A is a vertical cross-section view of an alternative configuration of a radiation detector unit according to an embodiment of the present disclosure.

FIG. 6B is a side elevation view of a detector module including a plurality of radiation detector units of FIG. 6A according to an embodiment of the present disclosure.

FIG. 7A is a side view of an alternative configuration of a radiation detector unit according to various embodiments of the present disclosure.

FIG. 7B is a top view of the radiation detector unit of FIG. 7A.

FIG. 8 is a side elevation view of a detector module including a plurality of radiation detector units according to an embodiment of the present disclosure.

FIG. 9A is a side cross-section view of a detector structure according to an embodiment of the present disclosure.

FIG. 9B is a horizontal cross-section view of a portion of the carrier board of the detector structure of FIG. 9A.

FIG. 10 is a side cross-section view of a detector structure that includes a plurality of resistive heating traces on and within the carrier board according to an embodiment of the present disclosure.

FIG. 11 is a horizontal cross-section view of a portion of a carrier board of a detector structure that includes a resistive heating trace according to an embodiment of the present invention.

FIG. 12A is a side cross-section view of an assembly including an ASIC located over a carrier board prior to mounting the ASIC to the carrier board according to an embodiment of the present disclosure.

FIG. 12B is a horizontal cross-section view of a portion of a carrier board illustrating an opening and a plurality of conductive vias extending through the carrier board according to an embodiment of the present disclosure.

FIG. 12C is a side cross-section view of the assembly of FIG. 12A following the application of an underflow material according to an embodiment of the present disclosure.

### DETAILED DESCRIPTION

Embodiments of the present disclosure provide detector structures, such as radiation detector units and radiation detector modules, and detector arrays formed by assembling the detector structures, and methods of manufacturing the same, the various aspects of which are described herein with reference to the drawings.

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims. Any reference to claim elements in the singular, for example, using the articles “a,” “an,” or “the” is not to be construed as limiting the element to the singular. The terms “example,” “exemplary,” or any term of the like are used herein to mean serving as an example, instance, or illustration. Any implementation described herein as an “example” is not necessarily to be construed as preferred or advantageous over another implementation. The drawings are not drawn to scale. Multiple instances of an element may be duplicated where a single instance of the element is illustrated, unless absence of duplication of elements is expressly described or clearly indicated otherwise.

FIG. ° A is a functional block diagram of an X-ray imaging system 100 in accordance with various embodiments. The X-ray imaging system 100 may include an X-ray source 110 (i.e., a source of ionizing radiation), and an energy discriminating photon counting radiation detector 120. The X-ray imaging system 100 may additionally include a patient support structure 105, such as a table or frame, which may rest on the floor and may support an object 10 to be scanned. In some embodiments, the object 10 may be a biologic subject (i.e., a human or animal patient). The support structure 105 may be stationary (i.e., non-moving) or may be configured to move relative to other elements of the X-ray imaging system 100, such as the X-ray source.

The X-ray source 110 is typically mounted to a gantry and may move or remain stationary relative to the object 10. The X-ray source 110 is configured to deliver ionizing radiation to the radiation detector 120 by emitting an X-ray beam 107 toward the object 10 and the radiation detector 120. After the X-ray beam 107 is attenuated by the object 10, the beam of radiation 107 is received by the radiation detector 120.

The radiation detector 120 may be controlled by a high voltage bias power supply 124 that selectively creates an electric field between an anode 128 and cathode 122 pair coupled thereto. In one embodiment, the radiation detector 120 includes a plurality of anodes 128 (e.g., one anode per pixel) and one common cathode 122 electrically connected to the power supply 124 and facing the X-ray source 110. The radiation detector 120 may include a detector material 125, such as a semiconductor material disposed between the anode 128 and cathode 122 and thus configured to be exposed to the electrical field therebetween. The semiconductor material may comprise any suitable semiconductor material for detecting X-ray radiation disposed between the anode 128 and cathode 122 and thus configured to be exposed to the electrical field therebetween. In various embodiments, the semiconductor material of the radiation detector 120 may comprise a II-VI semiconductor material, such as cadmium telluride, cadmium zinc telluride (i.e., CdZnTe or “CZT”), cadmium selenide telluride, and cadmium zinc selenide telluride. Other suitable semiconductor materials are within the contemplated scope of disclosure.

A detector application specific integrated circuit (ASIC) 130 (such as a detector readout integrated circuit (ROIC)), may be coupled to the anode(s) 128 of the radiation detectors 120. The detector ASIC 130 may receive signals (e.g., charge or current) from the anode 128(s) and be configured to provide data to and by controlled by a control unit 170. The signals received by the detector ASIC 130 may be in response to photon interaction events occurring within the radiation-sensitive semiconductor material of the detector material 125. Accordingly, the signals received by the detector ASIC 130 may be referred to as “event detection signals.” The radiation detector 120 may be segmented or configured into a large number of small “pixel” detectors 126. In various embodiments, the pixel detectors 126 of the radiation detector 120 and the readout circuit 130 are configured to output data that includes counts of photons detected in each pixel detector in each of a number of energy bins. Thus, radiation detectors 120 of various embodiments provide both two-dimensional detection information regarding where photons were detected, thereby providing image information, and measurements of the energy of the detected X-ray photons. A radiation detector 120 that is capable of measuring the energy of the X-ray photons impinging on the detector 120 may be referred to as an energy-discriminating radiation detector 120.

The control unit 170 may be configured to synchronize the X-ray source 110, the detector ASIC 130, and the high voltage bias power supply 124. The control unit 170 may be coupled to and operated from a computing device 160. Alternatively, the computing device 160 and the control unit 170 may be integrated together as one device.

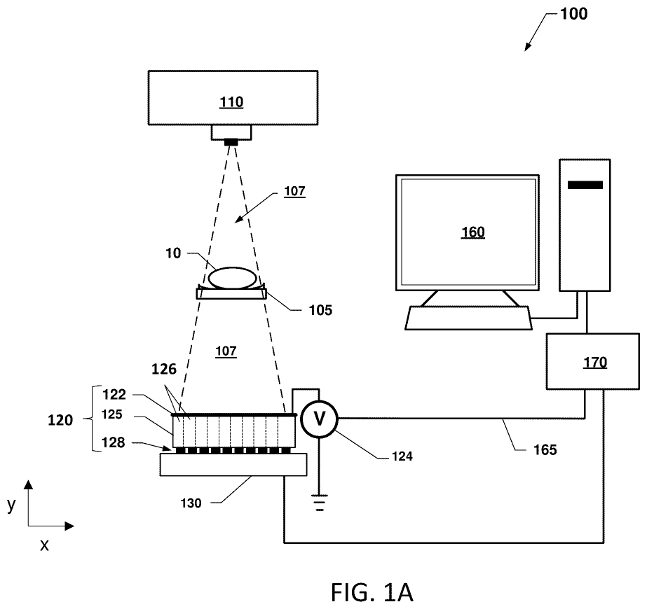

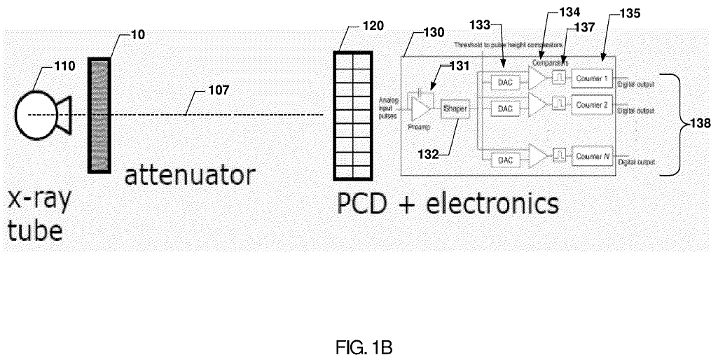

In some embodiments, the X-ray imaging system 100 may be a computed tomography (CT) imaging system. The CT imaging system 100 may include a gantry (not shown in FIG. 1A), which may include a moving part, such as a circular, rotating frame with the X-ray source 110 mounted on one side and the radiation detector 120 mounted on the other side. The radiation detector 120 may have a curved shape along its long axis (i.e., the x-axis direction in FIG. 1A) such that each of the pixel detectors along the length of the radiation detector may face towards the focal spot of the X-ray source 110. The gantry may also include a stationary (i.e., non-moving) part, such as a support, legs, mounting frame, etc., which rests on the floor and supports the moving part. The X-ray source 110 may emit a fan-shaped or cone-shaped X-ray beam 107 as the X-ray source 110 and the radiation detector 120 rotate on the moving part of the gantry around the object 10 to be scanned. After the X-ray beam 107 is attenuated by the object 10, the X-ray beam 107 is received by the radiation detector 120. The curved shape of the radiation detector 120 may allow the CT imaging system 100 to create a 360° continuous circular ring of the image of the object 10 by rotating the moving part of the gantry around the object 10.

For each complete rotation of the X-ray source 110 and the radiation detector 120 around the object 10, one cross-sectional slice of the object 10 may be acquired. As the X-ray source 110 and the radiation detector 120 continue to rotate, the radiation detector 120 may take numerous snapshots called “views”. Typically, about 1,000 profiles are taken in one rotation of the X-ray source 110 and the radiation detector 120. The X-ray source 110 and the detector 120 may slowly move relative to the patient along a horizontal direction (i.e., into and out of the page in FIG. 1A) so that the detector 120 may capture incremental cross-sectional profiles over a region of interest (ROI) of the object 10, which may include the entire object 10. The data acquired by the radiation detector 120 and output by the ASIC 130 may be passed along to the computing device 160 that may be located remotely from the radiation detector 120 via a connection 165. The connection 165 may be any type of wired or wireless connection. If the connection 165 is a wired connection, the connection 165 may include a slip ring electrical connection between any structure (e.g., gantry) supporting the radiation detector 120 and a stationary support part of the support structure, which supports any part (e.g., a rotating ring). If the connection 165 is a wireless connection, the radiation detector 120 may contain any suitable wireless transceiver to communicate data with another wireless transceiver that is in communication with the computing device 160. The computing device 160 may include processing and imaging applications that analyze each profile obtained by the radiation detector 120, and a full set of profiles may be compiled to form a three-dimensional computed tomographic (CT) reconstruction of the object 10 and/or two-dimensional images of cross-sectional slices of the object 10.

Various alternatives to the design of the X-ray imaging system 100 of FIG. 1A may be employed to practice embodiments of the present disclosure. X-ray imaging systems may be designed in various architectures and configurations. For example, an X-ray imaging system may have a helical architecture. In a helical X-ray imaging scanner, the X-ray source 110 and radiation detector 120 are attached to a freely rotating gantry. During a scan, a table moves the object 10 smoothly through the scanner, or alternatively, the X-ray source 110 and detector 120 may move along the length of the object 10, creating helical path traced out by the X-ray beam. Slip rings may be used to transfer power and/or data on and off the rotating gantry. In other embodiments, the X-ray imaging system may be a tomosynthesis X-ray imaging system. In a tomosynthesis X-ray scanner, the gantry may move in a limited rotation angle (e.g., between 15 degrees and 60 degrees) in order to detect a cross-sectional slice of the object 10. The tomosynthesis X-ray scanner may be able to acquire slices at different depths and with different thicknesses that may be reconstructed via image processing.

FIG. 1B illustrates components of an X-ray imaging system, including components within the detector ASIC 130 configured to count X-ray photons detected in each pixel detector within a set of energy bins. As used herein, the terms “energy bin” and “bin” refer to a particular range of measured photon energies between a minimum energy threshold and a maximum energy threshold. For example, a first bin may refer to counts of photons determined to have an energy greater than a threshold energy (referred to as a trigger threshold, e.g., 20 keV) and less than 40 keV, while a second bin may refer to counts of photons determined to have an energy greater than 40 keV and less than 60 keV, and so forth.

X-rays 107 from an X-ray source (e.g., X-ray tube) 110 may be attenuated by a target (e.g., an object 10, such as a human or animal patient) before interacting with the radiation detector material within the pixelated detector array 120. An X-ray photon interacting (e.g., via the photoelectric effect) with a pixelated radiation detector material generates an electron cloud within the material that is swept by an electric field to the anode electrode 128. The charge gathered on the anode 128 creates a signal (i.e., an above-described event detection signal) that is transmitted to the readout circuit 120 and integrated by a charge sensitive amplifier (CSA) 131. There may be a CSA 131 for each pixel detector (e.g., for each anode 128) within the pixelated X-ray detector 120. The voltage of the CSA output signal may be proportional to the energy of the X-ray photon. The output signal of the CSA may be processed by an analog filter or shaper 132.

The filtered output may be connected to the inputs of a number of analog comparators 134, with each comparator connected to a digital-to-analog converter (DAC) 133 that inputs to the comparator a DAC output voltage that corresponds to the threshold level defining the limits of an energy bin. The detector ASIC 130 may be configured so that after the CSA voltage has stabilized (after the dead time), that voltage may be between two voltage thresholds set by two DACs 133, which determines the output of the comparators 134. Outputs from the comparators 134 may be processed through decision gates 137, with a positive output from a comparator 134 corresponding to a particular energy bin (defined by the DAC output voltages) resulting in a count added to an associated counter 135 for the particular energy bin. Periodically, the counts in each energy bin counter 135 are output as signals 138 to the control unit 170.

The detector array of an X-ray imaging system may include an array of radiation detector elements, referred to herein as pixel detectors. The signals from the pixel detectors may be processed by a pixel detector circuit, which may sort detected photons into energy bins based on the energy of each photon or the voltage generated by the received photon. When an X-ray photon is detected, its energy is determined and the X-ray photon count for its associated energy bin is incremented. For example, if the detected energy of an X-ray photon is 24 kilo-electron-volts (keV), the X-ray photon count for the energy bin of 20-40 keV may be incremented. The number of energy bins may be three or more, such as four to twelve. In an illustrative example, an X-ray photon counting detector may have four energy bins: a first bin for detecting photons having an energy between 20 keV and 40 keV, a second bin for detecting photons having an energy between 40 keV and 60 keV, a third bin for detecting photons having an energy between 60 keV and 90 keV, and a fourth bin for detecting photons having an energy above 90 keV (e.g., between 90 keV and 120 keV). The greater the total number of energy bins, the better the material discrimination. The total number of energy bins and the energy range of each bin may be selectable by a user, such as by adjusting the threshold levels defining the limits of the respective energy bins in the ASIC 130 as shown in FIG. 1B.

In various embodiments, a radiation detector 120 for an X-ray imaging system 100 as described above may include a detector array including a plurality of pixel detectors 126 extending over a continuous two-dimensional (2D) detector array surface. The detector array (which is also known as a detector module system (DMS)) may include a modular configuration including a plurality of detector modules, where each detector module may include at least one radiation sensor (e.g., a detector material 125 including cathode and anode electrode(s) 122, 128 defining pixel detectors 126 as described above), at least one ASIC 130 electrically coupled to the at least one radiation sensor, and a module circuit board. The module circuit board may support transmission of electrical power, control signals, and data signals between the module circuit board and the at least one ASIC 130 and the at least one radiation sensor of the detector module, and may further support transmission of electrical power, control signals, and data signals between the module circuit board and the control unit 170 of the X-ray imaging system 100, other module circuit boards of the detector array, and/or a power supply for the detector array. A plurality of detector modules may be assembled on a common support structure, such as a detector array frame, to form a detector array.

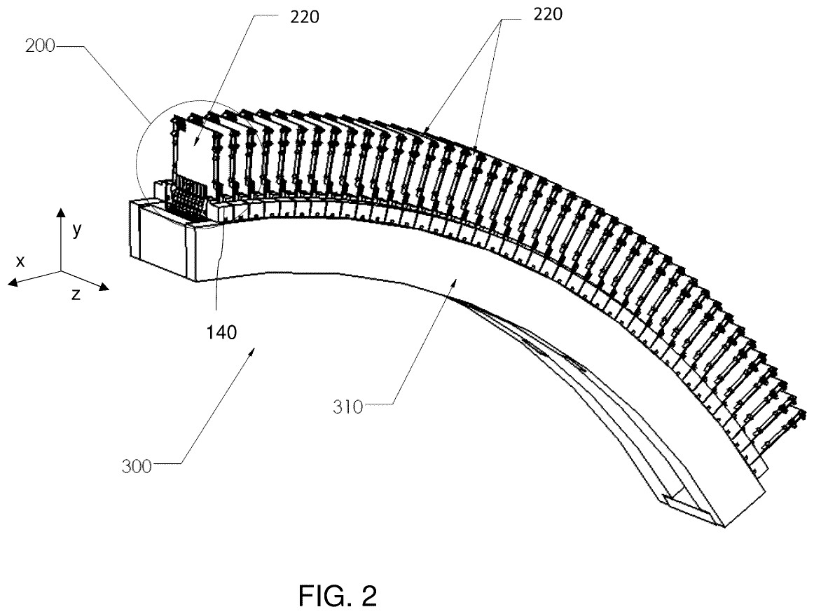

FIG. 2 is a perspective view of a detector array 300 for a computed tomography (CT) X-ray imaging system according to various embodiment of the present disclosure. The detector array 300 in this embodiment includes multiple detector modules 200 mounted on a detector array frame 310. The detector array frame 310 may be configured to provide attachment of a row of detector modules 200 such that physically exposed surfaces of the radiation sensors of the detector modules 200 collectively form a curved detection surface located within a cylindrical surface. The multiple detector modules 200 may be assembled such that radiation sensors attached to neighboring detector modules 200 abut each other, i.e., make direct surface contact with each other and/or include a gap between adjacent radiation sensors that is less than 3 mm, and/or less than 2 mm, and/or less than 1 mm in the x-direction. In some embodiments, the detector modules 200 may be mounted to the detector array frame 310 by attaching frame bars 140 of the detector modules 200 to the detector array frame 310 using suitable mechanical fasteners. The radiation sensors and ASICs 130 of each module 200 may be mounted over a first (i.e., front) surface of the frame bar 140. Each module 200 may also include a module circuit board 220 extending away from a rear surface of the frame bar 140. Major surfaces of the module circuit boards 220 of the detector modules 200 may face each other in the detector array 300.

In some embodiments, each of the detector modules 200 of a detector array 400 may be constructed from a set of radiation detector units, which may also be referred to as “mini-modules” or “submodules.” In some embodiments, each of the radiation detector units may include one or more radiation sensors coupled to a single ASIC 130. The radiation detector units according to various embodiments may be designed to minimize gaps between adjacent pairs of radiation detector units. Thus, a two-dimensional array of four side buttable radiation detector units forming a continuous detector surface may be provided without gaps, or with only minimal gaps, among the radiation detector units.

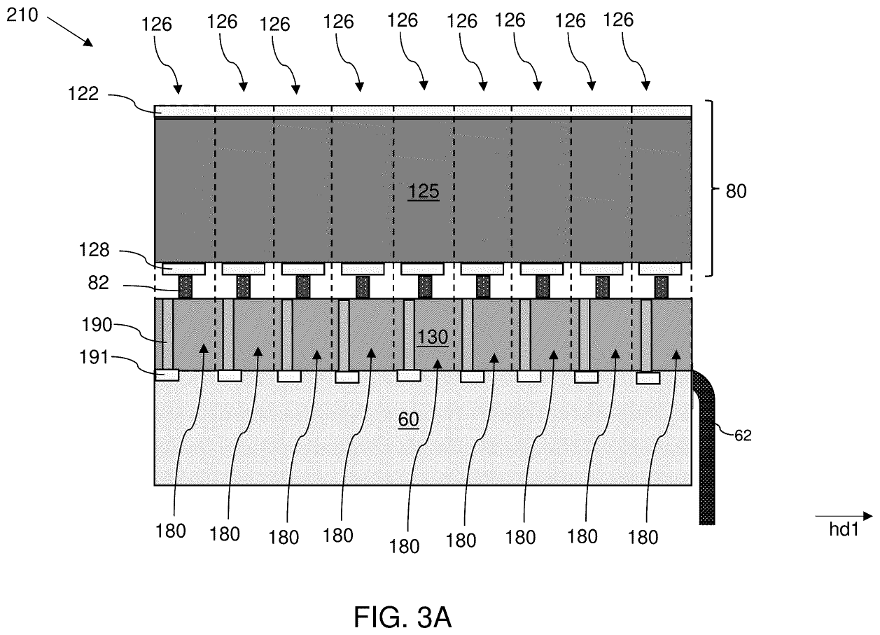

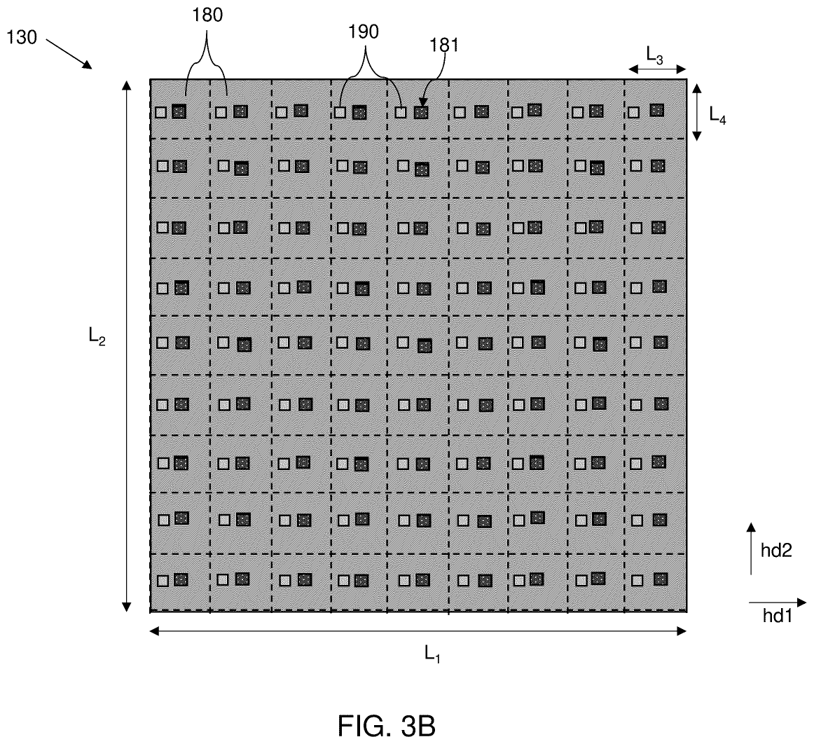

FIG. 3A is a vertical cross-sectional view of a radiation detector unit 210 according to one embodiment of the present disclosure. Referring to FIG. 3A, the radiation detector unit 210 includes a radiation sensor 80 coupled to an ASIC 130. The radiation sensor 80 may include an above-described detector material 125 having at least one cathode electrode 122 on a front side of the radiation sensor 80 and a plurality of anode electrodes 128 on a back side of the radiation sensor 80 defining an array of pixel detectors 126 as described above. As used herein, the “front side” of elements refers to the side that faces the incoming radiation, and the “backside” of elements refers to the side that is the opposite side of the front side.

The radiation sensor 80 may be directly mounted to the front side of the ASIC 130 via a plurality of bonding material portions 82. In other words, the radiation sensor 80 may be mechanically and electrically coupled to the ASIC 130 via the plurality of bonding material portions 82, and no interposer or similar intervening structural component for routing of electrical signals between the radiation sensor 80 and the ASIC 130 is located between the back side of the radiation sensor 80 and the front side of the ASIC 130. Directly mounting the radiation sensor(s) 80 to the front side of the ASIC 130 may provide a significant reduction in input node capacitance as compared to a radiation detector unit that includes an interposer located between the radiation sensor(s) 80 and the ASIC 130. For example, an embodiment radiation detector unit 210 having direct attachment of the radiation sensor(s) 80 to the ASIC 130 may provide an 80% or more reduction in the input node capacitance compared to an equivalent detector unit having an interposer (e.g., 0.2 pF vs. 1.0 pF). This may result in lower power consumption (e.g., 0.2 mW/channel compared to 0.8 mW/channel using an interposer) and lower equivalent noise charge (ENC) (e.g., 250 e− vs, 700 e− using an interposer).

The plurality of bonding material portions 82 may be arranged in an array, such as a rectangular array, having the same periodicity as the periodicity of the anode electrodes 128 on the back side of the radiation sensor 80. Thus, each bonding material portion 82 may electrically couple a respective anode electrode 128 of the radiation sensor 80 to the front side of the ASIC 130. In one non-limiting embodiment, the bonding material portions 82 may be composed of a conductive epoxy. Other suitable bonding materials, such as a low temperature solder material with under bump metallization, may be utilized to mount the radiation sensor 80 to the front side of the ASIC 130.

In various embodiments, the ASIC 130 may include an arrangement of electronic signal sensing channels and supporting logic circuitry in at least one monolithic component. The ASIC 130 may include an arrangement of circuit components (e.g., transistors, such as field effect transistors (FETs), resistors, capacitors, etc.) and associated interconnect structures located on and/or within a single supporting substrate, which may be a semiconductor material substrate (e.g., a silicon substrate). FIG. 3B is a plan view illustrating the front side of the ASIC 130 of the radiation detector unit 210 of FIG. 3A. In various embodiments, the dimensions of the ASIC 130 may generally correspond to the dimensions of the radiation sensor(s) 80 mounted over the front side of the readout circuit 130. In particular, the dimensions, L₁ and L₂, of the ASIC 130 along respective orthogonal horizontal directions hd1 and hd2 may be substantially equal (e.g., within +4%, such as +0-2%) to the dimensions of the radiation sensor(s) 80 mounted to the ASIC 130 along the same horizontal directions hd1 and hd2. In the embodiment illustrated in FIGS. 3A and 3B, a single radiation sensor 80 is mounted to the front side of the ASIC 130, although it will be understood that in other embodiments, multiple radiation sensors 80 may be mounted to the front side of the ASIC 130, such that the dimensions, L₁ and L₂, of the ASIC 130 along horizontal directions hd1 and hd2 may be substantially equal to the combined dimensions of the multiple radiation sensors 80 along the same horizontal directions hd1 and hd2. In embodiments in which multiple radiation sensors 80 having identical sizes are mounted to the front side of the ASIC 130, the dimensions L₁ and L₂, of the ASIC 130 may each be an integer multiple of the corresponding dimensions of the radiation sensors 80. The ASIC 130 and each of the radiation sensors 80 mounted thereto may have a rectangular periphery. This may enable any of the four peripheral sides of the radiation detector unit 210 to be abutted against a peripheral side of an adjacent radiation detector unit 210 upon assembly of multiple radiation detector units 210 in a two-dimensional detector array.

In various embodiments, the dimensions L₁ and L₂, of the ASIC 130 may each be greater than about 0.5 cm, such as at least about 1 cm. In some embodiments, the ASIC 130 may have at least one dimension (i.e., L₁ and/or L₂) that is at least about 4 cm, such as 8 cm or more (e.g., 8-16 cm), although greater and lesser dimensions for the ASIC 130 may be utilized.

Referring again to FIGS. 3A and 3B, the radiation sensor 80 may include array of contiguous pixel detectors 126 and the ASIC 130 may include a plurality of pixel regions 180 underlying each of the pixel detectors 126 of the radiation sensor 80, as indicated by the dashed lines in FIGS. 3A and 3B. A bonding material portion 82 may extend between each pixel detector 126 of the radiation sensor 180 and a corresponding pixel region 180 of the ASIC 130. Thus, as shown in FIG. 3B, each pixel region 180 of the ASIC 130 includes a contact region 181 in which a bonding material portion 82 contacts the front side of the ASIC 130. Metal interconnect structures (not shown in FIGS. 3A and 3B) on the front side of the ASIC 130 may electrically couple the contact regions 181 to the various circuit components (e.g., transistors, resistors, capacitors, etc.) of the ASIC 130. Each of the pixel regions 180 of the ASIC 130 may have dimensions, L₃ and L₄ along horizontal directions hd1 and hd2 that are substantially equal (e.g., within +4%, such as +0-2%) to the corresponding dimensions of the pixel detector 126 overlying the pixel region 180 of the ASIC 130. In some embodiments, the dimensions L₃ and L₄ of each pixel region 180 may be in a range of 250-500 μm, although greater and lesser dimensions are within the contemplated scope of disclosure. In one non-limiting embodiment, each of the pixel regions 180 of the ASIC 130 may be a 330 μm×330 μm square. In other embodiments, the pixel regions 180 may be rectangular-shaped in which the dimensions L₃ and L₄ are not equal. Each of the contact regions 181 of the pixel regions 180 may have dimensions along horizontal directions hd1 and hd2 that are each greater than about 50 μm, such as between 50 μm and 150 μm (e.g., ˜100 μm). In various embodiments, the plurality of pixel regions 180 may extend continuously over the entire area of the ASIC 130. In the illustrative embodiment shown in FIGS. 3A and 3B, the ASIC 130 includes a 9×9 matrix array of pixel regions 180 extending over the entire area of the ASIC 130, although it will be understood that An ASIC 130 having greater or lesser numbers of pixel regions 180 may be utilized in various embodiments.

Referring again to FIG. 3A, the radiation detector unit 210 may further include a carrier board 60 that is configured to route power supply to the ASIC 130 and to the at least one radiation sensor 80, control signals to the ASIC 130, and data signals (e.g., digital detection signals) generated by the ASIC 130. One or more cables 62, such as a flex cable assembly, may be attached to a respective side of the carrier board 60, and another end of each cable may be connected to a module circuit board 220 as shown in FIG. 2. The carrier board 60 may be a printed circuit board including an insulating substrate and printed interconnection circuits. In various embodiments, the ASIC 130 may be disposed over the carrier board 60 such that the back side of the ASIC 130 may contact the front side of the carrier board 60.

Referring again to FIGS. 3A and 3B, a plurality of through-substrate vias (TSVs) 190 may be provided in the ASIC 130. Each of the TSVs 190 may be located within a pixel region 180 of the ASIC 130. The TSVs 190 may include an electrically conductive material (e.g., a metal material, such as copper) that extends between the front side and the back side of the ASIC 130. In embodiments in which the ASIC 130 may be formed on and/or in a silicon substrate, the TSVs 190 may also be referred to as “through-silicon vias.”

Accordingly, electrical connections between the carrier board 60 and the ASIC 130 may be made through the back side of the ASIC 130 via the plurality of TSVs 190. In particular, each of the TSVs 190 may electrically contact a conductive trace 191 located on the front side of the carrier board 60, as schematically illustrated in FIG. 3A. This may obviate the need for wire bond and/or interposer connections between the front side of the carrier board 60 and the front side of the ASIC 130, which may help to minimize the footprint of the radiation detector unit 210. In various embodiments, outer periphery of the carrier board 60 may not extend beyond the outer periphery of the ASIC(s) 130 and radiation sensor(s) 80 located over the carrier board 60 so as to provide a radiation detector unit 210 that is buttable on all four sides.

The TSVs 190 may be fabricated by forming plurality of deep openings in the substrate using photolithographic patterning and an anisotropic etching process, performing thin film deposition of insulating, barrier and/or metallic seed layers within each of the openings, and filling the openings with a metallic fill material via a suitable deposition process, such as an electrodeposition process. A thinning process, such as a grinding or chemical-mechanical planarization (CMP) process, may be used to remove material from the backside of the substrate to expose the TSVs 190. In some embodiments, the substrate may be thinned to a thickness of less than 200 μm, such as 10 to 150 μm, for example, 50 to 100 μm. The TSVs 190 may be formed using a “TSV first” process in which the plurality of TSVs 190 may be formed through a semiconductor material substrate (e.g., a silicon wafer) prior to fabricating the electronic circuit components (e.g., transistors, capacitors, resistors, etc.) of the ASIC 130 via front end of the line (FEOL) semiconductor fabrication processes. In other embodiments, the TSVs 190 may be formed after FEOL processes are complete but prior to the formation of metal interconnect structures via back end of the line (BEOL) fabrication processes. In still further embodiments, the TSVs 190 may be formed using a “TSV last” process either during or following the completion of BEOL processes. “TSV last” fabrication may provide the highest degree of flexibility, as the ASIC 130 may be initially fabricated at a silicon foundry and then subsequently processed to form the TSVs 190.

Each of the TSVs 190 may have dimensions along horizontal directions hd1 and hd2 that are between about 1 μm and about 200 μm, although greater and lesser dimensions for the TSVs 190 may also be utilized. In one non-limiting embodiment, the dimensions of the TSVs 190 along horizontal directions hd1 and hd2 may be about 50 μm. As noted above, each of the TSVs 190 is located in a pixel region 180 of the ASIC 130 that underlies a pixel detector 126 of the radiation sensor 80. Thus, each of the TSVs 190 shares the pixel region 180 in which it is located with a contact region 181 that electrically couples the pixel region 180 to the overlying pixel detector 126 of a radiation sensor 80 via a bonding material portion 82. The TSVs 190 may be laterally spaced from the contact regions 181 to avoid electrically-shorting the bonding material portions 82 to the TSV 190. Metal interconnect structures (not shown in FIGS. 3A and 3B) on the front side of the ASIC 130 may electrically couple the TSVs 190 to the various circuit components (e.g., transistors, resistors, capacitors, etc.) of the ASIC 130. In the embodiment shown in FIGS. 3A and 3B, each of the pixel regions 180 of the ASIC 130 includes a single TSV 190, although it will be understood that in other embodiments, some or all of the pixel regions 180 may include multiple TSVs 190, and/or some of the pixel regions 180 may not include any TSVs 190. The total number of TSVs 190 may be sufficient to provide all the required electronic signaling (e.g., control signals and data output signals) between the ASIC 130 and the carrier board 60 as well as to provide all the required power to the ASIC 130.

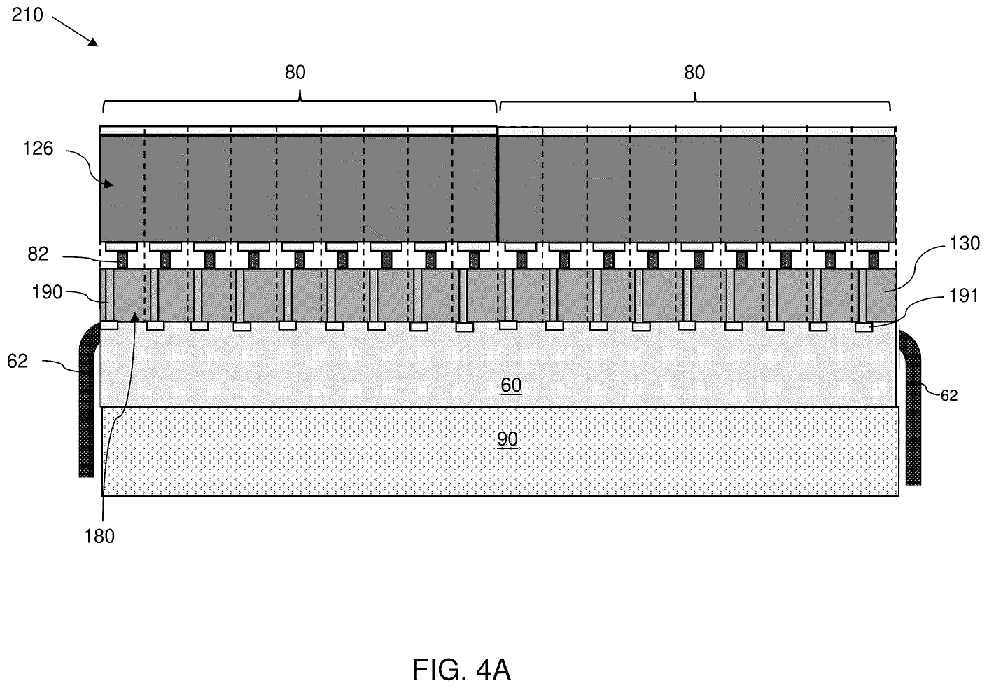

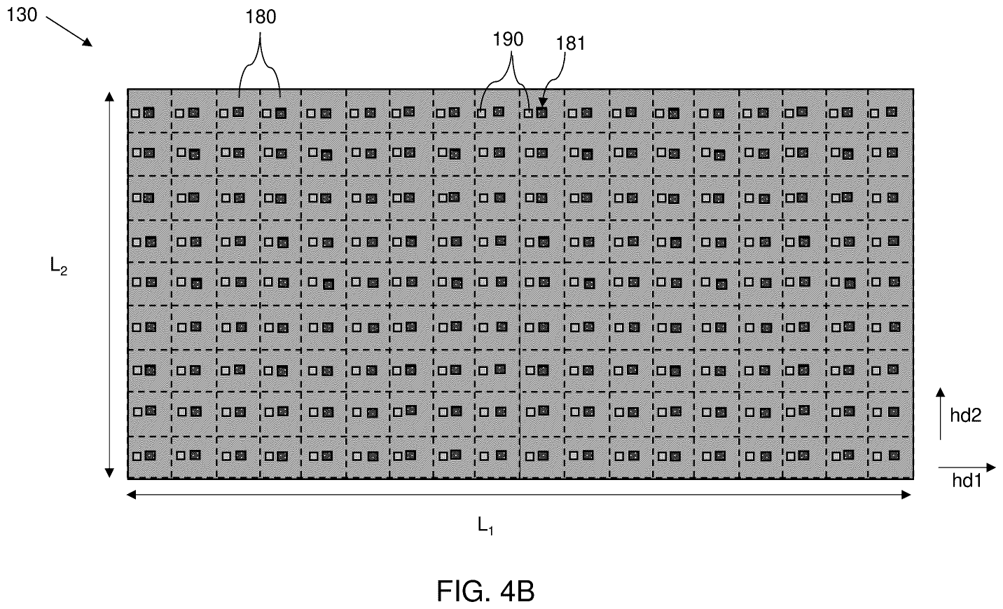

FIG. 4A is a vertical cross-section view of an alternative configuration of a radiation detector unit 210 according to an embodiment of the present disclosure. In this embodiment, a pair of radiation sensors 80 is directly mounted to an ASIC 130 via a plurality of bonding material portions 82. Thus, as shown in FIG. 4B, which is a plan view of the ASIC 130 of the radiation detector unit 210 of FIG. 4A, the ASIC 130 may include a length dimension L₁ along the first horizontal direction hd1 that is equal to the combined length dimensions of the pair of radiation sensors 80, and a width dimension L₂ along the second horizontal direction hd2 that is equal to the corresponding width dimensions of the radiation sensors 80. The ASIC 130 in this embodiment includes a contact region 181 and a TSV 190 within each of the pixel regions 180.

The radiation detector unit 210 of FIG. 4A includes a pair of cable connections 62 (e.g., flex cable assemblies) on opposite sides of the carrier board 60. In addition, the radiation sensors 80, the ASIC 130, and the carrier board 60 are mounted to a supporting substrate (e.g., a block) 90 as shown in FIG. 4A. The supporting substrate may include a high thermal conductivity material such as a metal (e.g., aluminum, copper, etc.). The supporting substrate 90 may function as a heat sink for the radiation detector unit 210. The supporting substrate 90 may be attached to the backside of the carrier board 60 using a thermally conductive adhesive such as a thermally conductive paste, and/or by mechanical connection structures (such as snap-in connectors, screws, and/or bolts and nuts).

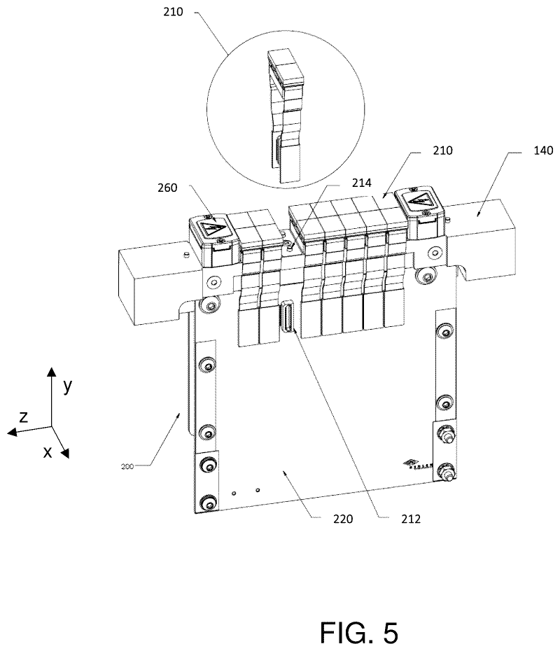

FIG. 5 is a perspective view of a detector module 200 including a plurality of radiation detector units 210 mounted to an above-described frame bar 140. Referring to FIG. 5, a row of radiation detector units 210 may be mounted to the front side of the frame bar 140. Engagement features 214 may optionally be provided on the front side of the frame bar 140 that may made with corresponding engagement features (not shown) on the backside of the supporting substrate 90 of each of the radiation detector units 210. End holders 260 may optionally be located at either end of the row of radiation detector units 210. A module circuit board 120 may be mechanically coupled to the frame bar 140 by suitable mechanical fastener(s). Each radiation detector unit 210 of the detector module may be electrically coupled to the module circuit board 220 by a flex cable assembly 62. In the embodiment shown in FIG. 5, the module circuit board 220 may include board-side connectors 212, where each board-side connector 212 may be connected to a connector, such as a snap-in connector 66, of a respective flex cable.

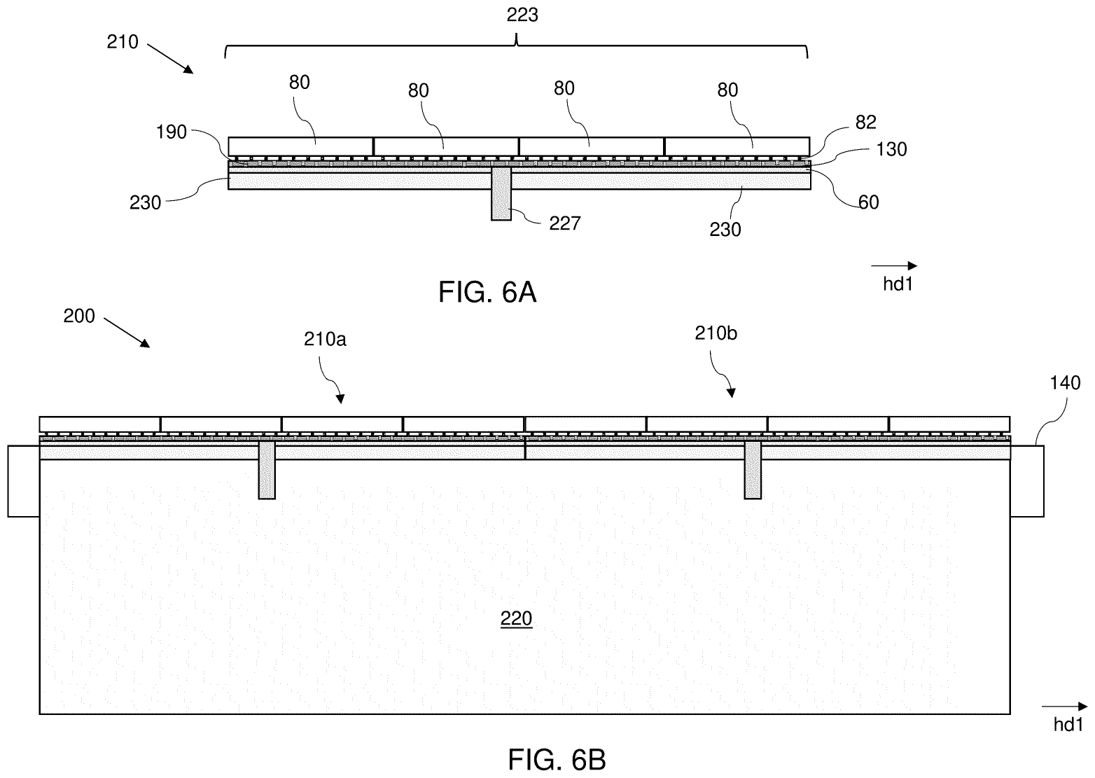

FIG. 6A is a vertical cross-section view of an alternative configuration of a radiation detector unit 210 according to an embodiment of the present disclosure. In the embodiment shown in FIG. 6A, a plurality of radiation sensors 80 are directly mounted to the front side of the ASIC 130 via bonding material portions 82. In the embodiment shown in FIG. 6A, the radiation detector unit 210 includes four radiation sensors 80 mounted over the front surface of the ASIC 130, although a greater or lesser numbers (e.g., between 1-8) of radiation sensors 80 may be mounted over the front surface of the ASIC 130. The radiation sensors 80 may abut one another along a first horizontal direction hd1 such that they provide a continuous radiation sensor area 223. The continuous radiation sensor area 223 may also extend across the entire width of the radiation detector unit 210 along a second horizontal direction that is perpendicular to the first horizontal direction hd1. The ASIC 130 may include a plurality of TSVs 190 located within pixel regions of the ASIC 130 underlying a pixel detector of a radiation sensor 80. The ASIC 130 may be located on a carrier board 60, which may be similar or identical to the carrier board 60 described above with reference to FIG. 3A. The carrier board 60 may be electrically coupled to the ASIC 130 via the plurality of TSVs 190. In some embodiments, an optional stiffening member 230 may be located on the backside of the carrier board 60 to provide increased mechanical support and stiffness to the radiation detector unit 210. At least one electrical connector 227 may be electrically connected to the radiation detector unit 210. In the embodiment shown in FIG. 6A, an electrical connector 227 is connected to the carrier board 60. The electrical connector 227 may be configured to route power supply to the ASIC 130, control signals to the ASIC 130, and data signals generated by the ASIC 130. In some embodiments, the electrical connector 227 may be a flex cable assembly as described above, although other suitable electrical connectors are within the contemplated scope of disclosure. Furthermore, although a single electrical connector 227 is shown in FIG. 6A, it will be understood that multiple electrical connectors 227 may be connected to the carrier board 60. For example, a high voltage electrical connector may be connected to the carrier board 60 and used to selectively provide a bias voltage to the radiation sensors 80 (e.g., to the cathodes of the radiation sensors) of the radiation detector unit 210.

FIG. 6B is a side elevation view of a detector module 200 including a plurality of radiation detector units 210a, 210b as described above with reference to FIG. 6A. Referring to FIG. 6B, a pair of above-described radiation detector units 210a, 210b may be arranged such that the peripheral edges 221 of the radiation detector units 210a, 210b abut against one another. This may increase the effective length of the continuous radiation sensor area 223 along the first horizontal direction hd1, which may correspond to the z-axis dimension in the assembled detector array as shown in FIG. 2. In various embodiments, the effective length of the continuous radiation sensor area 123 of the butted radiation detector units 210a, 210b may be at least about 6 cm, such as 8-40 cm, including between 12-24 cm (e.g., 14-18 cm). In some embodiments, the effective length of the continuous radiation sensor area 123 may be at least about 16 cm. A detector system including butted radiation detector units 210a, 210b and providing a continuous radiation sensor area 123 having an effective length in the z-axis direction of at least about 16 cm may be beneficial for a number of imaging applications, such as cardiac CT scanning. In the case of cardiac CT scans, for example, a larger detector length in the z-axis direction may enable the entire heart to be imaged in a single rotation about the patient (i.e., a single image slice).

The pair of detector modules 210a and 210b may be mounted over the front surface of a frame bar 140 that may function as a substrate for structurally holding the radiation detector units 210a and 210b in a butted configuration as shown in FIG. 6B. In some embodiments, the front surface of the frame bar 140 may include non-planar features, such as an outer lip or rim portion and a recessed flat central portion, to facilitate alignment of the radiation detector units 210a and 210b on the frame bar 140. In some embodiments, the frame bar 140 may be attached to the radiation detector units 210a and 210b using a thermally conductive adhesive, such as a thermally conductive paste, and/or by mechanical connection structures (such as snap-in connectors, screws, and/or bolts and nuts). The detector module 200 may further include a module circuit board 220 as described above with reference to FIG. 2. The detector module 200 may be attached to the frame bar 140 such that the module circuit board 220 may extend away from the rear side of the frame bar 140. Electrical connections between the respective radiation detector units 210a, 210b and the module circuit board 220 of the detector module 210 may be made via electrical connectors 227.

An above-described radiation detector unit 210 and/or detector module 200 that includes one or more radiation sensors 80 directly mounted to an ASIC 130 without an interposer or similar intervening structural component such that each pixel detector 126 is located over and is directly electrically connected to a corresponding input channel of the ASIC 130 may be referred to as a “direct attach” radiation detector unit 210 and/or a “direct attach” detector module 200. Exemplary embodiments of direct attach radiation detector units 210 and direct attach detector modules 200 that include a plurality of TSVs 190 that electrically connect the ASIC 130 to an underlying carrier board 60 are described in U.S. Provisional Patent Application No. 63/380,769, filed on Oct. 25, 2022, the entire teachings of which are incorporated by reference herein for all purposes.

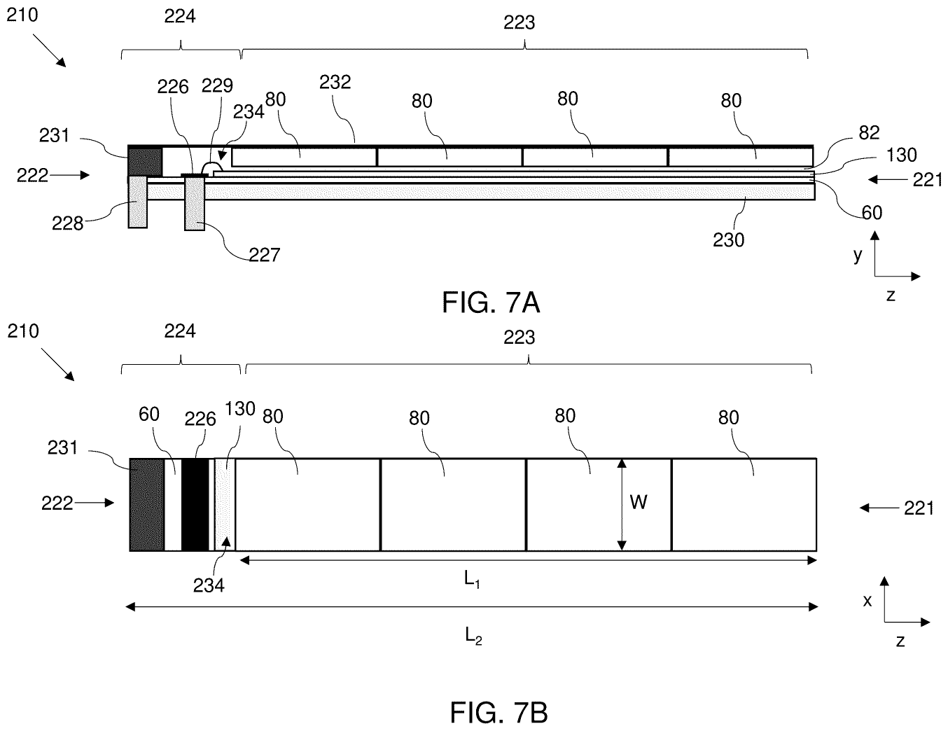

FIGS. 7A-7B are side and top views, respectively, of an alternative configuration of a direct attach radiation detector unit 210 according to various embodiments of the present disclosure. In the embodiment shown in FIGS. 7A and 7B, electrical connections between the carrier board 60 and the ASIC 130 may be made in a peripheral area 224 of the radiation detector unit such as wire bonds 229 extending between a bond pad region 226 on the front side of the carrier board 60 and a bond pad region 234 on the front side of the ASIC 130. Thus, in some embodiments, no conductive via structures, such as TSVs 190, may extend from the backside of the ASIC 130 through the semiconductor material substrate of the ASIC 130. In alternative embodiments, at least some of the electrical connections between the carrier board 60 and the ASIC 130 may be through the backside of the ASIC 130 (e.g. via TSVs 190). For example, a first set of connections (e.g., power and ground connections) between the carrier board 60 and the ASIC 130 may be made via TSVs through the backside of the ASIC 130 while other connections (e.g., data connections) between the carrier board 60 and the ASIC 130 may be made via wire bond connections 229 on the front sides of the carrier board 60 and the ASIC 130.

Referring again to FIGS. 7A and 7B, one or more radiation sensors 80 may be directly mounted to the front side of the ASIC 130 via bonding material portions 82, and no interposer or similar intervening structural component for routing of electrical signals between the radiation sensors 80 and the ASIC 130 is located between the backside of the one or more radiation sensors 80 and the front side of the ASIC 130. Thus, the radiation detector unit 210 may be considered a “direct attach” radiation detector unit 210. In the embodiment shown in FIGS. 7A and 7B, the radiation detector unit 210 includes four radiation sensors 80 mounted over the front surface of an ASIC 130, although a greater or lesser numbers (e.g., between 1-8) of radiation sensors 80 may be mounted over the front surface of the ASIC 130. The radiation sensors 80 may abut one another along the z-axis direction such that they provide a continuous radiation sensor area 223 extending along the z-axis direction. The continuous radiation sensor area 223 may extend to a first peripheral edge 221 of the radiation detector unit 210. The continuous radiation sensor area 223 may also extend across the entire width, W, of the radiation detector unit 210 along the x-axis direction, as shown in FIG. 4B. The radiation detector unit 210 may also include a peripheral area 224 that does not include any radiation sensors 80. The peripheral area 224 may be located between the continuous radiation sensor area 223 and a second peripheral edge 222 of the radiation detector unit 210 that is opposite the first peripheral edge 221. In various embodiments, a portion of the ASIC 120 including a bond pad region 234 may extend partway into the peripheral area 224 of the radiation detector unit 210 as shown in FIGS. 7A and 7B.

In various embodiments, the ASIC 130 may include an arrangement of electronic signal sensing channels and supporting logic circuitry in at least one monolithic component. The ASIC 130 may include an arrangement of circuit elements located on and/or within a single supporting substrate, which may be a semiconductor material substrate (e.g., a silicon substrate). The ASIC 130 may include core circuit blocks underlying respective radiation sensors 80 in the continuous radiation sensor area 223 of the radiation detector unit 210. The ASIC 130 may also include a peripheral circuit block in the peripheral area 224 of the radiation detector unit 210. A radiation sensor 80 may be bonded to each of the core circuit blocks of the ASIC 130 in the assembled radiation detector unit 100. The core circuit blocks may each include an array of bonding pads on the front surface of the ASIC 130 having a periodicity that corresponds to the periodicity of the pixel detectors of the radiation sensors 80, such that each of the bonding pads within a core circuit block of the ASIC 130 may be bonded to a respective pixel detector of a radiation sensor 80 via a bonding material portion 82. Each of the core circuit blocks may have length and width dimensions that correspond to the length and width dimensions of a radiation sensor 80 that is bonded to the core circuit block on the front side of the ASIC 130. Each of the core circuit blocks may also include additional circuit elements, such as electronic signal sensing channels and supporting logic circuitry. In some embodiments, each of the core circuit blocks of the ASIC 130 may include identical circuit elements in an identical layout as each of the other core circuit blocks of the ASIC 130. The peripheral circuit block of the ASIC 130 may include input/output (I/O) circuitry for the ASIC 130, including, for example, output bond pads for transmitting digitized radiation event detection signals from the ASIC 130, input bond pads for receiving control signals for the ASIC 130, and power pads for providing electrical power to the ASIC 130. The input bond pads, output bond pads and power pads may be located in a bond pad region 234 of the ASIC 130. In some embodiments, an ASIC 130 as described above may be fabricated using a photolithographic “stitching” process as described in U.S. patent application Ser. No. 18/158,695 filed Jan. 24, 2023, the entire teachings of which are incorporated herein by reference for all purposes.

Referring again to FIGS. 7A and 7B, the ASIC 130 may be located on a carrier board 60, which may be similar to the carrier board 60 described above with reference to FIGS. 3A, 4A and 6A. In some embodiments, an optional stiffening member 230 may be located on the backside of the carrier board 60 to provide increased mechanical support and stiffness to the radiation detector unit 210. The carrier board 60 and the optional stiffening member 230 may extend over the full length, L₂, of the radiation detector unit 210 between the first peripheral edge 221 and the second peripheral edge 222 of the radiation detector unit 210. At least one electrical connector 227, 228 may be electrically connected to the radiation detector unit 210. In the embodiment shown in FIG. 7A, a first electrical connector 227 may be electrically connected to a bond pad region 226 of the carrier board 60. At least one wire bond 229, which may be one or more reverse wire bonds, may electrically connect bond pads in the bond pad region 226 of the carrier board 60 with bond pads in a bond pad region 234 on the front side of the ASIC 130. The first electrical connector 227 and the wire bonds 229 may be configured to route power supply to the ASIC 130, control signals to the ASIC 130, and data signals generated by the ASIC 130. In some embodiments, the first electrical connector 227 may be a flex cable assembly as described above, although other suitable electrical connectors are within the contemplated scope of disclosure. Furthermore, although a single first electrical connector 227 is shown in FIG. 7A, it will be understood that multiple first electrical connectors 227 may be connected to the carrier board 60. A second electrical connector 228 may be a high voltage electrical connector that is used to selectively provide a bias voltage to the radiation sensors 80 (e.g., to the cathodes of the radiation sensors) of the radiation detector unit 210. The second electrical connector 228 may be electrically connected to an electrical filter assembly 231 that is configured to condition the voltage provided via the second electrical connector 228 to improve the stability of the bias voltage provided to the radiation sensors 80. As shown in FIG. 7A, a conductive member 232, which may comprise a metal foil sheet, may extend from the filter assembly 231 over the front side surfaces (e.g., cathodes) of the radiation sensors 80 to provide the bias voltage to the radiation sensors 80 (for purposes of clarity, the conductive member 232, the first and second electrical connectors 227, 228 and the wire bonds 229 are omitted in FIG. 7B).

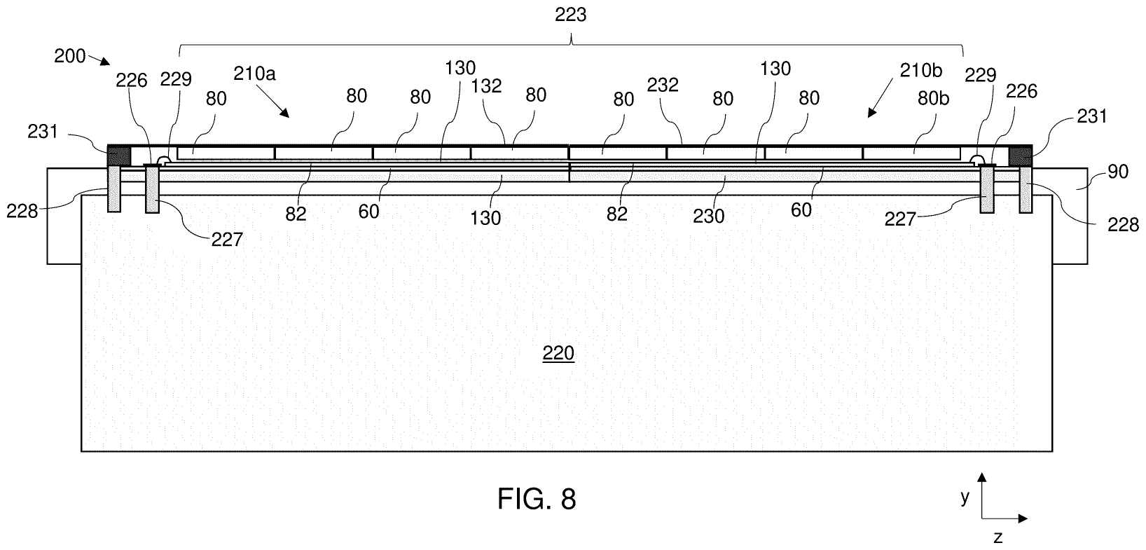

FIG. 8 is a side elevation view of a direct attach detector module 200 including a plurality of radiation detector units 210a, 210b as described above with reference to FIGS. 7A and 7B. Referring to FIG. 8, a pair of above-described direct attach radiation detector units 210a, 210b may be arranged such that the first peripheral edges 221 of the radiation detector units 210a, 210b abut against one another. This may increase the effective length of the continuous radiation sensor area 223 along the z-axis direction as shown in FIG. 8. The pair of detector modules 210a and 210b may be mounted over the front surface of a frame bar 90 that may function as a substrate for structurally holding the radiation detector units 210a and 210b in a butted configuration as shown in FIG. 8. In some embodiments, the front surface of the frame bar 90 may include non-planar features, such as an outer lip or rim portion and a recessed flat central portion, to facilitate alignment of the radiation detector units 210a and 210b on the frame bar 90. In some embodiments, the frame bar 90 may be attached to the radiation detector units 210a and 210b using a thermally conductive adhesive, such as a thermally conductive paste, and/or by mechanical connection structures (such as snap-in connectors, screws, and/or bolts and nuts). The detector module 200 may further include a module circuit board 220 as described above with reference to FIGS. 2, 5 and 6B. The detector module 200 may be attached to the frame bar 90 such that the module circuit board 220 may extend away from the rear side of the frame bar 90. Electrical connections between the respective radiation detector units 210a, 210b and the module circuit board 220 of the detector module 210 may be made via electrical connectors 227 and 228.

In some embodiments, the effective length of the continuous radiation sensor area 223 of the butted radiation detector units 210a and 210b along the z-axis may be at least about 6 cm, such as 8-40 cm, including between 12-24 cm (e.g., 14-18 cm). In some embodiments, the effective length of the continuous radiation sensor area 223 may be at least about 16 cm. A detector system including detector modules 200 as shown in FIG. 8 and providing a continuous radiation sensor area 223 having an effective length in the z-axis direction of at least about 16 cm may be beneficial for a number of imaging applications, such as cardiac CT scanning. In the case of cardiac CT scans, for example, a larger detector length in the z-axis direction may enable the entire heart to be imaged in a single rotation about the patient (i.e., a single image slice).

A radiation detector unit 210 and/or a detector module 200 as described above may have non-uniformities in the responses of different pixel detectors 126 of the array of pixel detectors 126. These non-uniformities in the detector pixel response may include variations in pixel performance with respect to a number of key detector parameters, including count uniformity (how uniform is the output count rate of the pixel detectors across different areas of the detector in response to the same incident photon flux?) and count stability (how stable is the behavior of the pixel detectors over time?). It is desirable to minimize variations in count uniformity and count stability within a detector system. In the case of direct attach radiation detector units 210 and direct attach detector modules 200 as described above, the behavior of the detector pixels, both in terms of count uniformity and count stability, may be sensitive to the temperature in both the radiation sensor 80 and the ASIC 130. Therefore, thermal non-uniformities within the radiation detector unit 210 and/or detector module 200, including both spatial and temporal thermal non-uniformities, should be minimized.

There are a number of causes of thermal non-uniformities in a radiation detector unit 210 and/or a detector module 200 as described above. For example, certain operating conditions and/or use cases of the imaging system may give rise to temperature transients. These may include changes in the power usage by the ASIC 130 due to changing flux of the incident radiation, start-up transients (e.g., when the system goes from an idle to a ready state), mission mode transients such as rapid changes in the X-ray tube power, room temperature control, and CT gantry thermal lag. Within the radiation detector unit 210 or detector 200, the ASIC 130 is the predominant source of thermal energy. However, there may be non-uniformities in the thermal energy produced by the ASIC 130. For example, the ASIC 130 may have certain regions or “hot spots” that produce more thermal energy than other regions of the ASIC 130. These may include, for example, regions of the ASIC 130 that include voltage regulator circuitry (e.g., a low-dropout (LDO) regulator) and/or regions of the ASIC 130 that include input/output circuitry (e.g., low voltage differential signaling (LVDS) circuitry). Further, as discussed above, the semiconductor substrate of the ASIC 130 is often thinned (e.g., to less than 200 μm, such as 50-100 μm), which may constrain the flow of heat laterally within the ASIC 130. This may further contribute to localized “hot spots” in the radiation detector unit 210 or detector module 200.

Further, the design of the radiation detector unit 210 and/or detector module 200 may contribute to thermal non-uniformities. In the case of a radiation detector unit 210 and/or detector module 200 having a relatively larger detector length along one direction (e.g., the z-axis direction as shown in FIGS. 7A-8, for example), there may be thermal non-uniformities resulting from airflow patterns over and/or around the detector during an imaging scan that may provide a greater degree of cooling in some regions of the detector than in others. In addition, in a detector module 200 as shown in FIG. 8, the ends of the module 200 (i.e., the opposite ends of the frame bar 90) may be connected to a gantry frame, detector chassis or other large structural element that may facilitate heat transfer from the ends of the module 200 into the adjacent structural element(s). However, the central portion of the module 200 may be located a relatively large distance from the adjacent structural element(s) on the opposite sides of the module 200 which may limit the amount of heat that may be transferred out from the central portion of the module 200. Accordingly, the central portion of the module 200 may have a higher temperature than the end portions of the module 200.

Various embodiments include a detector structure, such as an above-described radiation detector unit 210 and/or an above-described detector module 200, that includes at least one radiation sensor 80 directly attached (i.e., without the use of an interposer or similar intervening structure) to the front side of an ASIC 130 and a carrier board 60 located on the back side of the ASIC 130. The carrier board 60 may include one or more thermal management features that may reduce temperature non-uniformities in the detector structure.

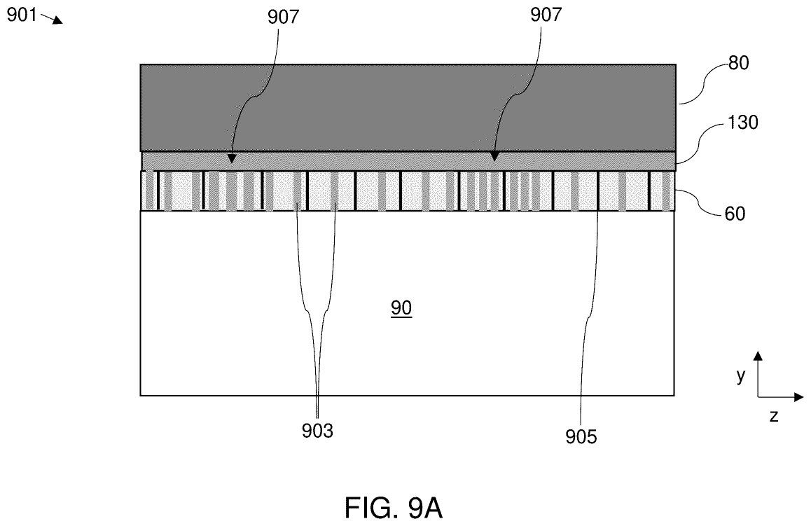

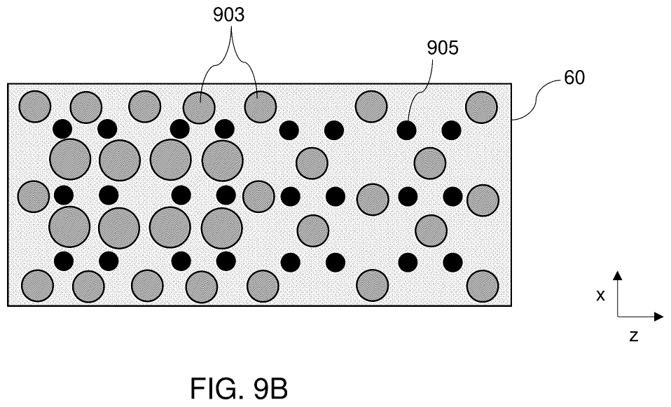

FIG. 9A is a side cross-section view of a detector structure 901 according to an embodiment of the present disclosure. FIG. 9B is a horizontal cross-section view of a portion of the carrier board 60 of the detector structure 901 of FIG. 9A. Referring to FIGS. 9A and 9B, a plurality of thermal vias 903 may extend through the carrier board 60. The thermal vias 903 may help to dissipate heat generated by the ASIC 130 and direct the heat away from the radiation sensors 80, which may be thermally-sensitive components. In some embodiments, the thermal vias 903 may be configured direct heat downwards and into an underlying frame bar 90. In other embodiments, the carrier board 60 may provide sufficient mechanical support for the ASIC 130 and the radiation sensor(s) 80, such that the frame bar 90 may be omitted. The thermal vias 903 may include openings extending through the carrier board 60. A thermally-conductive fill material, such as a metal (e.g., copper, etc.) may fill at least a portion of the openings. For example, sidewalls of the openings may be coated with a metal material. Alternatively or in addition, the entire volumes of the openings may be filled with a metal material.

In some embodiments, the carrier board 60 may further include electrical interconnect structures extending over and/or through the carrier board 60. For example, the carrier board 60 may be a printed circuit board (PCB) formed of an organic laminate structure and including conductive traces and vias extending over and within the laminate structure. In other embodiments, the carrier board 60 may be composed of a ceramic material that may have conductive interconnect structures (e.g., traces and/or vias) extending on and/or within the ceramic material. Other suitable materials for the carrier board 60 are within the contemplated scope of discussion.

FIGS. 9A and 9B also illustrate a plurality of conductive vias 905 extending through the carrier board 60. The conductive vias 905 may be electrically coupled to an above-described conductive trace 191 and/or a bonding pad (not shown in FIGS. 9A and 9B) on a front side surface of the carrier board 60, which may in turn be electrically coupled to the ASIC 130 by a TSV 190 as shown in FIGS. 3A-4B and/or by a wire bond connection 229 as shown in FIGS. 7A-8. Thus, electrical signals (e.g., power and/or data signals) may be passed through the carrier board 60 between the ASIC 130 and an above-described module board 220 or other components of the imaging system.

In various embodiments, the thermal vias 903 within the carrier board 60 may be non-functional structures, meaning that unlike the conductive vias 905 of the carrier board 60, the thermal vias 903 may not carry electrical power or data. Each of the thermal vias 903 may have a horizontal cross-section dimension (e.g., a diameter) that is greater than the corresponding horizontal cross-section dimension of the conductive vias 905. In some embodiments, the thermal vias 903 may have a horizontal cross-section dimension that is at least two times greater, such as at least five times greater, including up to ten times greater or more, than the horizontal cross-section dimensions of the conductive vias 905. In one non-limiting example, the conductive vias 905 may have a horizontal cross-section dimension (e.g., diameter) that is between about 20-50 μm and the thermal vias 903 may have a horizontal cross-section dimension (e.g., diameter) that is greater than 100 μm, such as 200 to 1000 μm. In some embodiments, the thermal vias 903 and the conductive vias 905 may be composed of the same material(s) (e.g., copper). Alternatively, the thermal vias 903 and the conductive vias 905 may be composed of different materials.

In various embodiments, the thermal vias 903 may have a non-uniform distribution within the carrier board 60. The non-uniform distribution of the thermal vias 903 may be configured to improve the equalization of temperature across different regions of the detector structure 901. In some embodiments, the size and/or density (i.e., number of thermal vias 903 per unit area) of the thermal vias 903 may be selected to compensate for a non-uniform temperature profile of the overlying ASIC 130. For example, a greater number of thermal vias 903 and/or relatively-larger sized thermal vias 903 may be located proximate to above-described “hot spots” in the ASIC 130, such as regions of the ASIC 130 that include voltage regulator circuitry (e.g., a low-dropout (LDO) regulator) and/or regions of the ASIC 130 that include input/output circuitry (e.g., low voltage differential signaling (LVDS) circuitry).

FIG. 9A illustrates an embodiment that includes a greater density of thermal vias 903 and/or larger sized thermal vias 103 in areas of the carrier board 60 that underlie “hot spots” 907 in the ASIC 130. Similarly, FIG. 9B illustrates a greater density of thermal vias 903 and relatively larger-sized (e.g., greater width or diameter) thermal vias 903 located on the left-hand side portion of the carrier board 60 than on the right-hand side portion of the carrier board 60. In some embodiments, the thermal vias 103 may equalize the temperature profile within the detector structure 901 such that the temperature within the ASIC 130 and the radiation sensor(s) 80 of the detector structure 901 may be maintained within +1° C. of each other during operation.

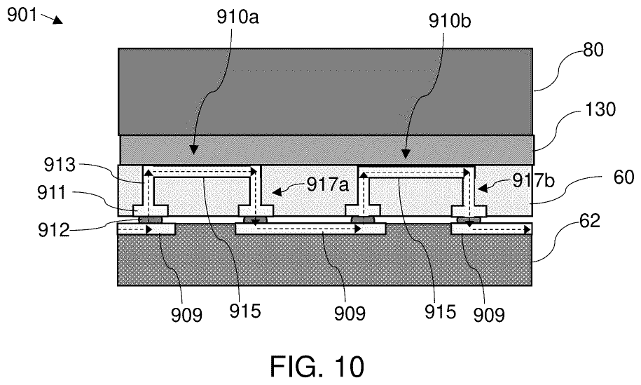

Further embodiments include detector structures 901 that include one or more heater elements configured to equalize a temperature profile within the detector structure 901. In some embodiments, the one or more heater elements may be located, at least in part, within a carrier board 60 of the detector structure 901. FIG. 10 is a side cross-section view of a detector structure 901 (e.g., an above-described radiation detector module 210 and/or detector module 200) that includes a plurality of resistive heating traces 915 on and/or within the carrier board 60. In some embodiments, the carrier board 60 may include a plurality of contiguous heater segments 917a, 917b on and/or within the carrier board 60. Each contiguous heater segment 917a, 917b may include a resistive heating trace 915 extending in a horizontal direction over and/or within the carrier board 60, and a pair of conductive vias 913 extending vertically through the thickness of the carrier board 60 and connecting the resistive heating trace 915 to an input connector (e.g., a bonding pad 911) and an output connector (e.g., a bonding pad 911). In some embodiments, each of the contiguous heater segments 917a and 917b may be electrically isolated from one another within the carrier board 60. The contiguous heater segments 917a and 917b may be electrically connected to one another in a daisy-chain configuration using one or more external connectors. In the exemplary embodiment of FIG. 10, the contiguous heater segments 917a and 917b may be connected to one another by an external cable (i.e., an above-described flex cable assembly 62) that may include a plurality of conductive traces 909 that connect the contiguous heater segments 917a and 917b in series. In other embodiments, the contiguous heater segments 917a and 917b a may be connected to one another by a circuit board, such as an above-described module board 220 that may be directly connected to the carrier board 60. In the exemplary embodiment of FIG. 10, conductive traces 909 on the flex cable assembly 62 may be electrically coupled to bonding pads 911 on the carrier board 60 via solder material portions 912. It will be understood that other mechanisms may be utilized for electrically coupling the flex cable assembly or other external connector to the input connector (e.g., a bonding pad 911) and the output connector (e.g., a bonding pad 911) of each contiguous heater segment 917a, 917b.

In some embodiments, the resistive heating traces 915 of the contiguous heater segments 917a, 917b may follow a tortuous or serpentine path along a horizontal plane over and/or within the carrier board 60. The widths of the resistive heating traces 915 and the conductive vias 913 may be less than the widths (e.g., diameters) of the thermal vias 903 described above with reference to FIGS. 9A and 9B (i.e., the thermal vias 903 and the heating traces 915 may be used in combination). Alternatively, in the embodiment of FIG. 10, the thermal vias 903 may be omitted. The conductive vias 913 and resistive heating traces 915 may be composed of a suitable electrically conductive material, such as a metal or a conductive ceramic-based material. In some embodiments, the conductive vias 913 and resistive heating traces 913 may be composed of a conductive material having a relatively high resistivity, such as a nickel-chromium alloy. Other suitable materials are within the contemplated scope of disclosure.

In various embodiments, the conductive vias 913 and restive heater traces 915 of the contiguous segments 917a and 917b may not carry data and/power signals between the carrier board 60 and the overlying ASIC 130. Rather, the conductive vias 913 and resistive heating traces 915 of the contiguous segments 917a and 917b may be configured to heat selected regions of the detector structure 901 including regions of the ASIC 130 and/or the radiation sensor(s) 80. A current may be provided as indicated by the dashed arrows in FIG. 10. The current may flow from a conductive trace 909 within the flex cable assembly 62 through a solder material portion 912 and into an input connector (i.e., a bonding pad 911) of a first contiguous heater segment 917a. The current may flow through a first conductive via 913, the resistive heating trace 915, and a second conductive via 913 of the first contiguous heater segment 917a to heat a first region 910a of the detector structure 901. The current may then flow from the output connector (i.e., a bonding pad 911) of the first contiguous heater segment 917a through a solder material portion 912 to a conductive trace 909 in the flex cable assembly 62 that may carry the current to a second contiguous heater segment 917b of the carrier board 60. The current may then flow through the second contiguous heater segment 917b to heat a second region 910b of the detector structure 901 and out of the carrier board 60 to another conductive trace 909 in the flex cable assembly.

The regions 910a and 910b of the detector structure 901 that include the contiguous heater segments 917a and 917b may correspond to regions 910a and 910b of the detector structure 901 that have a relatively lower temperature than other regions of the detector structure 901. Accordingly, by heating these relatively lower temperature regions 910a and 910b of the detector structure 901, a temperature profile within the detector structure 901 may be made more uniform. In some embodiments, the temperature within the detector structure 901 may be maintained within +1° C. (i.e., the coldest part of the detector structure 901 is within 1° C. of the hottest part of the same detector structure 901. For example, for an above-described radiation detector unit 210 and/or detector module 200 having a relatively larger detector length along one direction (e.g., the z-axis direction as shown in FIGS. 7A-8), the resistive heating traces 913 and 915 may be used to flatten the thermal profile non-uniformities across the z-axis length of the detector. Alternatively or in addition, the resistive heating traces 913 and 915 may be used dynamically on an as-needed basis, such as to pro-actively heat the detector structure 901 to reduce temperature changes and/or temperature variations in the detector structure 901 during an imaging scan.

In various embodiments, different detector structures 901 (e.g., radiation detector units 201 and/or detector modules 200) may have different configurations of heating elements, which may depend, for example, on different thermal profiles of the different detector structures 901, the location(s) of particular detector structures 901 within a larger area array (e.g., DMS), or other factors. For example, different detector structures 901 may include carrier boards 60 having a greater or lesser number of heating elements (e.g., contiguous heater segments 917a and 917b in FIG. 10), and/or having heating elements in different locations than in other carrier boards 60. In some embodiments, each carrier board 60 may have a similar or identical configuration of heating elements (e.g., contiguous heater segments 917a and 917b as shown in FIG. 10), but only a subset of the heating elements may be actively coupled to a current source in particular detector structures 901. Further, although the embodiment of FIG. 10 illustrates the resistive heating traces 915 extending over the front side surface of the carrier board 60 adjacent to the ASIC 130, in other embodiments the resistive heating traces 915 may be embedded within the carrier board 60.

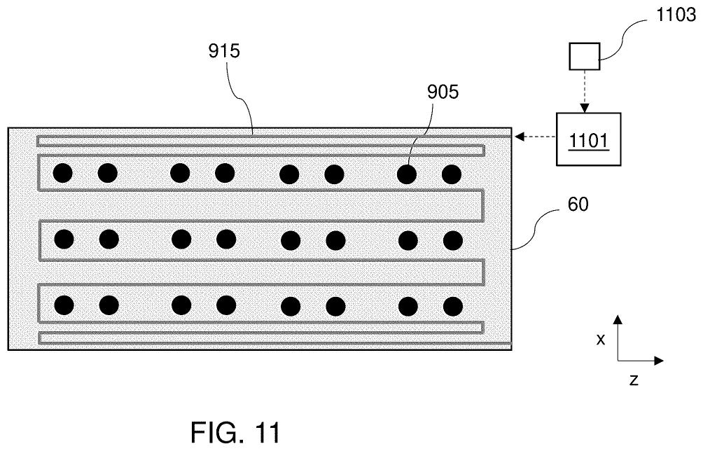

FIG. 11 is a horizontal cross-section view of a portion of a carrier board 60 of a detector structure 901 that includes a resistive heating trace 915 according to an embodiment of the present invention. The resistive heating trace 915 may extend in a horizontal direction (i.e., parallel to the front side surface of the carrier board 60 and the back side surface of an ASIC 130 mounted to the front side of the carrier board 60). The resistive trace 915 may follow a serpentine path over and/or within the carrier board 60 and around structures such as the conductive vias 905 as shown in FIG. 11 and/or above-described thermal vias (not shown in FIG. 11). In some embodiments, one or more resistive heating traces 915 may be embedded within a laminate structure carrier board 60, such as PCB, such as within a dedicated layer of the laminate structure carrier board 60. For example, a resistive heating trace 915 may be formed by lithographically patterning a conductive layer (e.g., a metallic laminate cladding) in a suitable pattern during the fabrication of the laminate structure carrier board 60 to provide a carrier board 60 having an embedded resistive heating trace 915 as shown in FIG. 11. The resistive heating trace 915 may be used to heat portions of a detector structure 901 including the carrier board 60 to provide a more uniform thermal profile in the detector structure 901.

Further embodiments include systems and methods for performing dynamic thermal management in an above-described detector structure 901 (e.g., a radiation detector unit 210 and/or detector module 200). This may enable improved equalization of temperature in the detector structure 901 during transient thermal conditions, such as during startup conditions, rapid changes in ASIC 130 power requirements, and the like.

Referring again to FIG. 11, a control system 1101 may be used to control a current within a heating element of a carrier board 60, such as the resistive heating traces 913 and 915 shown in FIGS. 10 and 11. The control system 1101 may include or may be coupled to a switch or relay that may be used to selectively turn on or off the current within the heating element 915. In some embodiments, the control system 1101 may also include or be coupled to a variable resistor or other device(s) that may be used to adjust an amount of current flowing through the heating element 915 at a given time. The control system 1101 may be configured to implement a control loop that may dynamically control the amount of current flowing within the heating element 915, and thereby control the amount of heat generated by the heating element 915, at a particular time. In some embodiments, the control system 1101 may include or may be coupled to a sensor 1103 configured to measure an operating variable of detector structure 901 and to control the current flow in the heating element 915 in response to a measured operating variable. For example, the operating variable measured by the sensor 1103 may include the instantaneous current drawn by the ASIC 103 and/or the temperature of the ASIC 103, which may indicate changes in the power usage by the ASIC 103. In response to a detected change in the power usage by the ASIC 130, the control system 1101 may be configured to control the current to the heating element 915 to implement a change in the power used by the heating element 915. The change in the heating element 915 power may be proportional to the change in the ASIC 130 power in some embodiments. In other embodiments, the sensor 1103 may include one or more temperature sensors that may measure the temperature of the ASIC 130. The control system 1101 may be configured to control the current through the heating element 915 to maintain the ASIC 130 at a constant temperature, such as within ±1° C. In some embodiments, the control system 1101 may include a logic circuit or embedded controller. In some embodiments, the control system 1101 may be located on and/or in the carrier board 60. In other embodiments, the control system 1101 may be located on another component, such as in the ASIC 130 or on a separate circuit board (e.g., a module board 220).

In other embodiments, rather than a control sequence using real-time monitoring of one or more operating variables, the control system 1101 may be configured to implement one or more pre-determined, pre-calibrated control sequences (e.g., at system startup or at other times) to provide a dynamically changing heating profile of the detector structure 901 in the temporal domain that provides improved temperature equalization during different operating conditions of the detector structure 901.

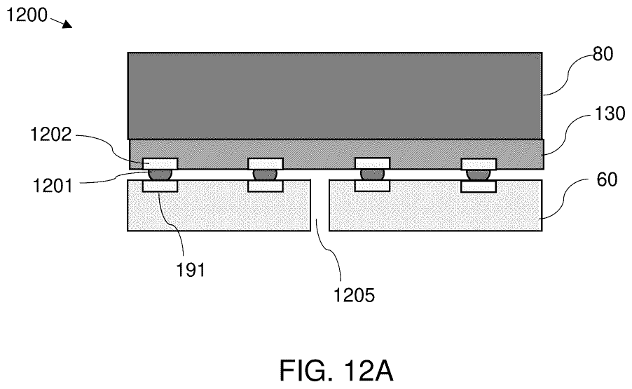

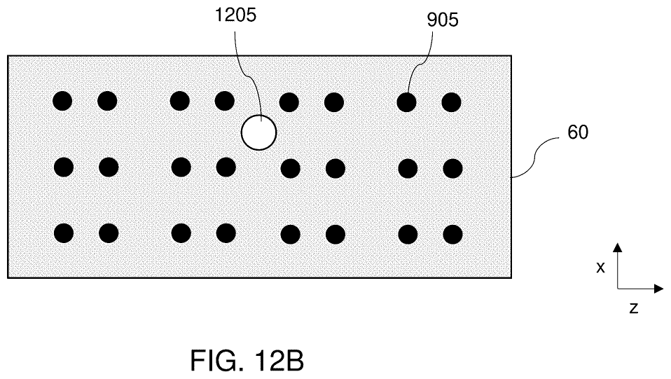

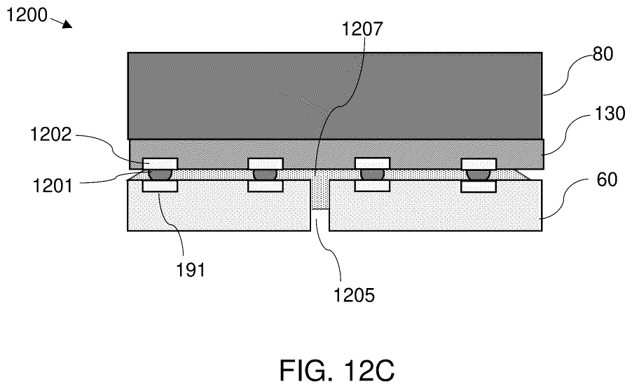

Further embodiments include detector structures 901 and methods of fabricating detector structures 901 that include at least one radiation sensor 80 mounted over the front side of an ASIC 130 and a carrier board 60 mounted to the back side of the ASIC 130. The carrier board 60 may include one or more features to improve the manufacturability of the detector structure 901. FIG. 12A is a side cross-section view of an assembly 1200 including an ASIC 130 located over a carrier board 60 prior to mounting the ASIC 130 to the carrier board 60. The embodiment of FIG. 12A illustrates a radiation sensor 80 mounted over the front side of the ASIC 130. In other embodiments, the ASIC 130 may be mounted to the carrier board 60 prior to or simultaneously with the mounting of one or more radiation sensors 80 to the ASIC 130. Referring again to FIG. 12A, a plurality of bonding material portions 1201 (e.g., solder balls) may be disposed between bonding structures 191 (e.g., bonding pads) on the front side surface of the carrier board 60 and bonding structures 1202 (e.g., bonding pads) on the back side surface of the ASIC 130. The bonding structures 1202 on the back side surface of the ASIC 130 may be coupled to above-described TSVs 190 (not shown in FIG. 12A).

In a detector structure 901, such as a radiation detector module 201 or a detector module 200 described above, in order to provide three-or-four side buttability when multiple detector structures 901 are assembled into a larger area array, the peripheral edges of the ASIC 130 may be coincident with peripheral edges of the carrier board 60 on at least two sides of the detector structure 901, and in some cases on three sides or on all four sides of the detector structure 901. This may present a challenge during the mounting of the ASIC 130 to the carrier board 60 to ensure that the alignment between the ASIC 130 and the carrier board 60 is maintained throughout the entire mounting process, which may include a solder reflow process. There may be an additional challenge in instances in which an underflow material is provided within the gap between ASIC 130 and the carrier board 60 and around the solder connections, since applying the underflow material from along the sides/edges of the assembly may result in overflow of the underfill material along the side surfaces of the detector structure 901.

Referring again to FIG. 12A, in some embodiments, one or more openings 1205 may be provided through the carrier board 60. FIG. 12B is a horizontal cross-section view of a portion of the carrier board 60 illustrating an opening 1205 and a plurality of conductive vias 905 extending through the carrier board 60 according to an embodiment of the present disclosure. The one or more openings 1205 may extend through the carrier board 60 between the front side and the back side of the carrier board 60 (i.e., the opening(s) 1205 may include unobstructed void areas between the front and back sides of the carrier board 60). Although a single opening 1205 is shown in FIGS. 12A and 12B, it will be understood that more than one opening 1205 may be present in the carrier board 60. The one or more openings 1205 may be in addition to one or more thermal management features in the carrier board 60, such as thermal via(s) 903 described above with reference to FIGS. 9A-9B and/or the resistive heating element 913, 915 described above with reference to FIGS. 10 and 11.

In some embodiments, the one or more openings 1205 may be fluidly coupled to a vacuum source (not illustrated in FIGS. 12A and 12B) during the process of mounting the ASIC 130 to the carrier board 60. The vacuum source may be operated to apply a negative pressure through the one or more openings 1205 that may help to maintain alignment of the ASIC 130 and the carrier board 60 during a bonding process (e.g., a solder reflow process) that bonds the ASIC 130 to the carrier board 60.

Following the bonding of the ASIC 130 to the carrier board 60, an underflow material may optionally be provided in the gap between the back side surface 130 of the ASIC 130 and the front side surface of the carrier board 60 and laterally surrounding the bonding material portions 1201 (e.g., solder connections). At least a portion of the underflow material may be applied through one or more openings 1205 extending through the carrier board 60. The opening(s) 1205 may include the same opening(s) 1205 used to apply the negative pressure during the bonding process, or may be different opening(s) 1205. The underflow material may include a suitable insulating material, such as an insulating epoxy material, and may be applied through the one or more openings 1205 using a suitable application process. FIG. 12C is a side cross-section view of the assembly 1200 following the application of the underflow material 1207. In some embodiments, a portion of the underflow material 1207 may be located within the one or more openings 1205 in the carrier board 60.

The devices of the embodiments of the present disclosure can be employed in various radiation detection systems including computed tomography (CT) imaging systems. Any direct conversion radiation sensors may be employed such as radiation sensors employing Si, Ge, GaAs, CdTe, CdZnTe, and/or other similar semiconductor materials.

The radiation detectors of the present embodiments may be used for medical imaging, such as in Low-Flux applications in Nuclear Medicine (NM), whether by Single Photon Emission Computed Tomography (SPECT) or by Positron Emission Tomography (PET), or as radiation detectors in High-Flux applications as in X-ray Computed Tomography (CT) for medical applications, and for non-medical imaging applications, such as in baggage security scanning and industrial inspection applications.

While the disclosure has been described in terms of specific embodiments, it is evident in view of the foregoing description that numerous alternatives, modifications and variations will be apparent to those skilled in the art. Each of the embodiments described herein can be implemented individually or in combination with any other embodiment unless expressly stated otherwise or clearly incompatible. Accordingly, the disclosure is intended to encompass all such alternatives, modifications and variations which fall within the scope and spirit of the disclosure and the following claims.

1. A detector structure, comprising:
an application specific integrated circuit (ASIC);
at least one radiation sensor located over a front surface of the ASIC; and
a carrier board located over a back surface of the ASIC and comprising a plurality of thermal vias extending through the carrier board.

2. The detector structure of claim 1, wherein each thermal via comprises an opening extending between a front surface and a back surface of the carrier board that is at least partially filled with a thermally conductive material.

3. The detector structure of claim 1, wherein a cross-section dimension of each of the thermal vias is at least 100 μm.

4. The detector structure of claim 1, wherein at least one of a size of the thermal vias or a density of the thermal vias varies in different regions of the carrier board.

5. The detector structure of claim 4, wherein at least one of a size or a density of the thermal vias is greater in regions of the carrier board that underlie regions of the ASIC that generate relatively more heat than in regions of the carrier board that underlie regions of the ASIC that generate relatively less heat.

6. The detector structure of claim 5, wherein regions of the regions of the ASIC that generate relatively more heat comprise regions of the ASIC containing voltage regulator circuitry or input/output circuitry.

7. The detector structure of claim 1, wherein the carrier board further comprises a plurality of conductive vias extending within the carrier board, wherein a cross-section dimension of each of the thermal vias is greater than two times the cross-section dimension of each of the conductive vias.

8. The detector structure of claim 7, wherein:
the cross-section dimension of each of the thermal vias is greater than ten times the cross-section dimension of each of the conductive vias; and
at least some of the conductive vias within the carrier board are electrically connected to the ASIC and none of the thermal vias are electrically connected to the ASIC.

9. The detector structure of claim 1, wherein the at least one radiation sensor is directly attached to the front surface of the ASIC without an interposer routing signals between at least one radiation sensor and the ASIC.

10. The detector structure of claim 9, wherein the ASIC is electrically coupled to the front side of the carrier board via at least one of:
a plurality of through-substrate vias extending through a substrate of the ASIC, and
a plurality of wire bonds extending between a bond pad region on the front side of the carrier board and a bond pad region on the front side of the ASIC.

11. The detector structure of claim 1, wherein the plurality of thermal vias are configured to maintain a temperature of the ASIC that is within ±1° C. of a temperature of the at least one radiation sensor.

12. The detector structure of claim 1, wherein the carrier board further comprises at least one resistive heating element located on or within the carrier board.

13. A detector structure, comprising:
an application specific integrated circuit (ASIC);
at least one radiation sensor located over a front surface of the ASIC; and
a carrier board located over a back surface of the ASIC, wherein the carrier board comprises at least one resistive heating element located on or within the carrier board.

14. The detector structure of claim 13, wherein the at least one resistive heating element comprises a resistive heating trace that extends in a direction that is parallel to the back surface of the ASIC.

15. The detector structure of claim 14, wherein the resistive heating trace follows a tortuous or serpentine path over or within the carrier board.

16. The detector structure of claim 13, wherein the at least one resistive heating element comprises a plurality of contiguous heater segments, each segment comprising a resistive heating trace that extends in a direction that is parallel to the back surface of the ASIC and a pair of conductive vias coupled to the resistive heating trace and extending within the carrier board along a direction that is perpendicular to the resistive heating trace, wherein each of the segments are electrically isolated from one another within the carrier board.

17. The detector structure of claim 16, further comprising an external connector coupled to the carrier board that electrically connects multiple continuous heater segments in series.

18. The detector structure of claim 13, wherein:
the at least one resistive heating element is configured to heat one or more regions of the detector structure that have a relatively lower temperature than other regions of the detector structure to equalize a temperature profile within the detector structure; and
the at least one resistive heating element is configured to heat one or more regions of the detector structure such that a temperature within different regions of the detector structure is maintained within ±1° C.

19. The detector structure of claim 13, further comprising a control system coupled to the at least one resistive heating element and configured to adjust a current flowing through the at least one resistive heating element in response to an operating variable of the detector structure.

20. The detector structure of claim 19, wherein the operating variable comprises at least one of a current in the ASIC or a temperature in the detector structure.

21. A detector structure, comprising:
an application specific integrated circuit (ASIC);
at least one radiation sensor located over a front surface of the ASIC; and
a carrier board located over a back surface of the ASIC, wherein the back surface of the ASIC is mounted to a front surface of the carrier board via a plurality of bonding material portions, and wherein the carrier board includes at least one opening extending through the carrier board.

22. The detector structure of claim 21, wherein peripheral side surfaces of the ASIC are coincident with peripheral side surfaces of the carrier board on at least two side surfaces of the ASIC.

23. The detector structure of claim 21, further comprising an underflow material located within a gap between the back surface of the ASIC and the front surface of the carrier board, wherein a portion of the underflow material is located within the one or more openings in the carrier board.

24. A method of fabricating a detector structure, comprising:
aligning an ASIC over a carrier board such that at least two peripheral side surfaces of the ASIC are coincident with peripheral side surfaces of the carrier board and a plurality of bonding material portions are disposed between the ASIC and the carrier board;
applying a negative pressure through an opening extending through the carrier board; and
bonding the ASIC to the carrier board using the bonding material portions.

25. The method of claim 24, wherein the bonding material portions comprise solder material portions and bonding the ASIC to the carrier board comprises performing a solder reflow process while the negative pressure is applied through the opening extending through the carrier board.

26. The method of claim 24, further comprising bonding at least one radiation detector to a front surface of the ASIC, and applying an underfill material through the opening extending through the carrier board and into a gap between the ASIC and the carrier board.

27. An X-ray imaging system, comprising:
a radiation source configured to emit an X-ray beam; and
a detector array including a plurality of detector structures of claim 1 that form a continuous detector surface and that are configured to receive the X-ray beam from the radiation source through an intervening space configured to contain an object therein.

28. The X-ray imaging system of claim 27, wherein the X-ray imaging system comprises a photon-counting computerized tomography (PCCT) imaging system comprising an image reconstruction system including a computer configured to run an automated image reconstruction algorithm on event detection signals generated by the detector modules of the detector array.
